(19) United States
(12) Patent Application Publication
(10) Pub. No.: US 2007/0041914 A1
(43) Pub. Date: Feb. 22, 2007

(54) Inhibition of bacterial deposition on oral surfaces

(75) Inventors: Abdul Gaffar, Princeton, NJ (US); Thomas J. Boyd, Metuchen, NJ (US)

Correspondence Address: COLGATE-PALMOLIVE COMPANY, 909 RIVER ROAD, PISCATAWAY, NJ, 08855, US

(73) Assignee: Colgate-Palmolive Company

(21) Appl. No.: 11/205,879

(22) Filed: Aug. 17, 2005

(51) Int. Cl. A61K 8/81

(52) U.S. Cl. 424/49; 424/56; 424/48

(57) ABSTRACT

The invention is directed to an oral composition that contains an amphoteric surfactant and an homo- or copolymer comprising an ethylenic monomer and related methods. The ethylenic monomer includes a phosphonate group. The monomer may be represented by the structure of Formula (I): 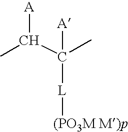 wherein
one of A and A′ is a hydrogen atom and the other is selected from a hydrogen atom or a group —(X)ₙRₘ; wherein n is i\selected from 0 or 1; provided that when n is 0, m is 1, and when n is 1, m is an integer of 1 to 3; X is selected from an oxygen atom, a sulfur atom, a nitrogen atom, a phosphorous atom, and silicon atom; R is independently a hyuderogen atom or an arganic radical; L is selected from a bond and a linking group; M and M′ are independently selected from a hydrogen atom, an alkali metal, and an ammonium ion, or together form an alkaline earth metal or other divalent element; and p is 1 or 2, provided that when L is a bond, p is 1.

### BACKGROUND OF THE INVENTION

A number of disease conditions are associated with the action of bacteria in the oral cavity. For example, gingivitis, an inflammation of infection of the gums and alveolar bone, is caused by toxins and other materials secreted by plaque forming bacteria, which include Actinomyces viscosus.

In addition, plaque provides loci for calculus or tartar formation. Periodontitis can develop when unremoved plaque hardens into calculus (tartar), which can affect the periodontal ligaments. As plaque and tartar continue to build up, the gums begin to recede, leading to continued infection and potentially loss of teeth. To prevent or treat these diseased conditions, antibacterial agents have been incorporated into oral care compositions such as toothpastes and mouthwashes. A multitude of materials have previously been proposed and used for controlling plaque, calculus, tartar, gingivitis and related disease conditions.

These conventional compositions may be effective. However, given the disparate medical, therapeutic and economic needs of oral care patients, it is nevertheless desirable to provide compositions and methods that are similarly effective against the deposition and attachment of bacteria on oral surfaces, to, e.g., prevent or reduce plaque formation and other diseases and disorders facilitated by oral bacteria.

### BRIEF SUMMARY OF THE INVENTION

The invention is directed to an oral composition that contains an amphoteric surfactant and an homo- or copolymer comprising an ethylenic monomer. The ethylenic monomer includes a phosphonate group. The monomer may be represented by the structure of Formula (I): 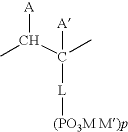
wherein

one of A and A′ is a hydrogen atom and the other is selected from a hydrogen atom or a group —(X)ₙRₘ; wherein n is i\selected from 0 or 1; provided that when n is 0, m is 1, and when n is 1, m is an integer of 1 to 3; X is selected from an oxygen atom, a sulfur atom, a nitrogen atom, a phosphorous atom, and silicon atom; R is independently a hyuderogen atom or an arganic radical;

L is selected from a bond and a linking group;

M and M′ are independently selected from a hydrogen atom, an alkali metal, and an ammonium ion, or together form an alkaline earth metal or other divalent element; and

p is 1 or 2, provided that when L is a bond, p is 1.

Also included are methods of inhibiting the deposition of bacteria and methods of promoting and maintaining systemic health by reduction of inflammation in the oral cavity through use of the oral composition.

### DETAILED DESCRIPTION OF THE INVENTION

The invention relates to compositions and methods for inhibiting bacterial deposition on oral surfaces. In particular, the invention relates to the use of compositions including phosphonic acid polymers and/or copolymers in combination with amphoteric surfactants to inhibit the depositions and/or attachment of oral bacteria to oral surfaces, thereby inhibiting plaque formation. Such compositions may be applied to the oral surfaces neat or may include other active agents upon or post-application.

The invention provides an oral composition containing a combination of an amphoteric surfactant and a homo- or co-polymer comprising an ethylenic monomer that comprises phosphonate groups. The composition may take any form suitable for administration to the oral cavity, including a dentifrice, mouthrinse, lozenge, chewing gum, confectionary, suspension, tablet, powder, paste, or gel. Illustratively, the amphoteric surfactant may be a betaine surfactant and the hom-co-polymer is a copolymer of vinylphosphonic acid. In preferred embodiments, the compositions further contain a phenol-containing compound.

The homo- or co-polymer may be any known or to be developed in the art, and includes polymers that contain ethylenic monomers comprising phosphonate group(s) (hereinafter “EMP”) solely or which contain EMP(s) that are copolymerized with other monomers. In such circumstances, it is preferable that the copolymer contains the EMP and the other monomer(s) is a ratio of a about 1:1, about 2:1 about 5:1 or about 10:1. Illustratively, the homo- or co-polymer includes a copolymer of vinylphosphonic acid and up to 50% mol of one or more non-fluorinated unsaturated monomers other than vinylphosphonic acid.

It may be preferred that the EMP is of a structure represented by Formula (I):

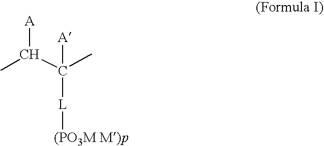

In Formula (I), at least one of A and A′ is a hydrogen atom and the other is a hydrogen atom or a group —(X)ₙRₘ, wherein n is independently 0 or 1, provided that when n is 0, m is 1 and when n is 1, m is independently an integer of 1 to 3. “X” represents an atom independently selected from an oxygen atom, a sulfur atom, a nitrogen atom, a phosphorous atom, and a silicon atom. The groups represented by R are each independently an hydrogen atom and/or an organic hydrocarbon radical, substituted or unsubstituted, preferably a C₁₋₅₀ organic radical or a C₅-C₁₈ organic radical. M and M′ are independently each selected from a hydrogen atom, an alkali metal, an ammonium ion, and/or together form a divalent alkaline earth element or other divalent cation.

The group represented by L may be a bond or a linking group, preferably an alkylene group (containing aliphatic carbon only) or alkyleneimino group (containing aliphatic nitrogen in addition to aliphatic carbon). In some embodiments it may be preferable that the linking group L contains: 1 to 12 total atoms of carbon and a nitrogen atom; 1 to 8 atoms of carbon and a nitrogen atom; or 1 to 6 total atoms of carbon and nitrogen. The group represented by p is 1 or 2, provided that when L is a bond, p is 1.

More preferred homo- or copolymers for use in the inventive compositions include those containing a plurality of phosphonate groups. In one embodiment, the polymer is a homo- or copolymer that comprises a plurality of repeating units of Formula I. For example, the polymer may be selected from the substituted or unsubstituted hydrocarbon radicals: alkyl, cycloalkyl, alkenyl, acyl, alkoxy, alkylthio, alkylsulfoxy, alkylsulfonyl, alkylamino, dialkylamino, dialkylphosphinyl, dialkylphosphinoxy trialkylsilyl radicals; benzyl, benzoyl, benzyloxy, benzylthio, benzylsulfoxy, benzylsulfonyl, benzylamino, benzoylamido, phenyl, phenoxy, phenylthio, phenylsulfoxy, phenylsulfonyl, phenylamino, phenylacetamido, xylyl, pyridyl and furanyl.

In one embodiment wherein the homo- or co-polymer contains at least one EMP of Formual (I), n may be 0, and the R groups may be independently selected from hydrogen, C₁₋₆ alkyl, C₃₋₆ cycloalkyl, phenyl and benzyl radicals.

Illustratively, the homo- or copolymer is a homopolymer wherein A in formula (I) is —(X)ₙ(R)ₘ, n is 0, m is 1 and R is a C₁₋₆ alkyl or phenyl group, and A′ is hydrogen. Where R is a methyl group, such a homopolymer is poly(1-phosphonopropene) or a salt thereof. Alternatively, where R is a phenyl group the homopolymemr is poly(β-styrenephosphonic acid) or a salt thereof.

The EMP can be present in its phosphonic acid form, where M and M′ are each hydrogen, or as a salt (including partial salt) thereof, wherein, at least one monomer at least one of M and M′ is alkali metal, typically sodium or potassium, or ammonium; or together M and M′ are an alkaline earth element (such as calcium) or other divalent element.

In one embodiment the EMP is a homopolymer of vinylphosphonic acid, or a salt (including partial salt) thereof. Such a compound is described herein as a “polyvinylphosphonate” and can be prepared by any process known in the art.

The polymer may have any molecular weight and such may be varied within the composition depending of the nature of the specific formulation and the end benefit desired. For example, the polymer may have an average molecular weight of at least about 1,000, preferably about 1,000 to about 100,000, about 5,000 to about 100,000, about 10,000 to about 100,000, about 15,000 to about 100,000, about 20,000 to about 100,000, about 25,000 to about 100,000 or about 25,000 to about 90,000. In one embodiment the average molecular weight is not less than about 22,000, for example about 22,000 to about 90,000, about 22,000 to about 70,000 or about 25,000 to about 35,000. In another embodiment the average molecular weight is not greater than about 20,000, for example about 5,000 to about 20,000 or about 5,000 to about 15,000.

Alternatively, the polymer may be a copolymer of (a) units having the molecular configuration of units derived from vinylphosphonic acid; and (b) units derived from vinylphosphonyl fluoride. A ratio of (a) units to (b) units from about 2:1 to about 25:1 is preferable.

The (a) units in the copolymer may be depicted as having the structural formula:

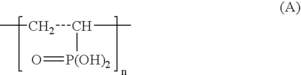

(a) n being a numerical value representing the number of (a) units.

The (b) units may be depicted as having the structural formula: 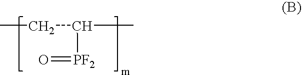
m “being a numerical value representing the number of (b) units in the copolymer”.

The (a) and (b) units may be randomly distributed in the copolymer molecule and that the copolymer may also contain minor proportions, derived from other ethylenically unsaturated monomers which, in type and amount, are non-toxic and preferably do not interfere with the desired anti-plaque and anti-gingivitis activities of the copolymer. Other such monomers may, for example, include olefins such as ethylene, propylene, isopropylene, butylene and isobutylene; vinyl lower alkyl ethers such as vinyl methyl-, ethyl- and isobutyl ethers; alpha,beta-unsaturated carboxylic acids and their lower alkyl and substituted lower alkyl esters such as acrylic, methacrylic, aconitic, maleic and fumaric acids and their methyl, ethyl, isobutyl and dimethylaminoethyl esters; allyl alcohol and acetate; vinyl and vinylidene halides; vinyl lower alkanoic acid esters such as vinyl acetate and butyrate; acrylamide and methacrylamide and N-lower alkyl and N,N-di(lower alkyl)-substituted derivatives thereof; other vinyl phosphonyl halides, and the like.

The (a) and (b) copolymers of this invention should preferably have a number average molecular weight of about 2,000, to about 50,000, more preferably about 3,500 to about 16,000. They may be prepared by polymerizing a mixture of vinyl phosphonyl chloride, as precursor of the (a) units, and vinyl phosphonyl fluoride as precursor of the (b) units, along with optional other monomers, under substantially anhydrous conditions in the presence of a free radical catalyst, and then mixing the resulting copolymer with water to hydrolytically convert the vinyl phosphonyl chloride units in the copolymer to vinyl phosphonic acid (a) units.

In another embodiment, the phosphonate polymers useful in the compositions of the invention comprise a homo- or copolymer of vinylphosphonic acid. In preferred embodiments, the vinylphosphonic acid co- or homopolymer has a molecular weight of about 4,000 to 9,100, more preferably about 6,000 to 8,900, determined as a number average molecular weight by gel permeation chromatography. Suitable polymers are prepared according to known methods by polymerizing the vinylphosphonyl dichloride under substantially anhydrous conditions in the presence of a free radical catalyst. After polymerization, the resulting polymer is rinsed with water to hydrolyze the vinyl phosphonic dichloride units to phosphonic acid units. The resulting polymer in free acid form is desirably covered to a salt form by neutralization, including partial neutralization, with a basic material containing an orally acceptable cation such as alkali metal, ammonium, organic amines, alkaline earth, etc. Copolymers containing vinylphosphonic acids may also contain minor proportions, that is preferably less than about 50% by weight, more preferably less than about 10% by weight, and more preferably about 2% by weight of repeating unites derived from other non-fluorinated ethylenically unsaturated monomers. Non-limiting examples of such non-fluorinated monomers are given above.

In various embodiments, phosphonate polymers of the invention contain recurring units of Formula I, wherein L is an alkylene or alkyleneimino connecting group. Representative polymers where L is alkylene include sodium poly(butene-4,4-diphosphonate) having units of the formula (II):

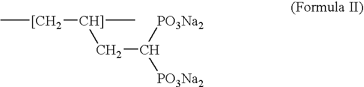

In similar manner, an illustrative polymer wherein B is alkyleneimino is poly-(allylbis(phosphonoethyl)amine) having units of the formula (III):

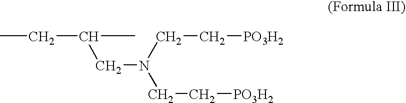

Compositions of the invention also contain an amphoteric surfactant. Amphoteric surfactants include those that those, depending on pH, take on either a negative or a positive charge. Suitable amphoteric surfactants include without limitation derivatives of C₈₋₂₀ aliphatic secondary and tertiary amines having an ionic group such as carboxylic, sulfate, sulfonate, phosphate, or phosphonate.

Betaine surfactants may be preferred. In one embodiment, betaine surfactants are derivatives of trimethylglycine(betaine). They may be characterized by the following structure represented by formula (IV):

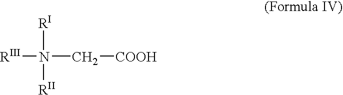

Where R^{I} and R^{II} are independently hydrogen or lower alkyl such as C₁₋₄ alkyl, preferably methyl. R^{III} is a hydrophobic group containing 6 or more, preferably 8 or more, and most preferably 12 or more carbon atoms. In one embodiment, R^{III} is an alkyl group and the amphoteric surfactant is an alkyl betaine. A non-limiting example is lauryl betaine. In another embodiment, R^{III} is an alkamidoalkyl group and the amphoteric surfactant is an amidoalkyl betaine. A non-limiting example is cocoamidopropyl betaine.

In preferred embodiments, the compositions further contain one or more phenol-containing compounds. In various embodiments, the phenol-containing compounds may function as flavors and/or antibacterial agents. They are characterized by having a benzene ring with a hydroxyl group directly attached. The other positions on the phenol ring can be substituted with a variety of groups such as C₁₋₈ alkyl, hydroxyl, alkoxy, and halogen. Phenol-containing compounds also include halogenated diphenyl ether compounds such as triclosan and diphenol materials such as honokiol and magnolol.

Non-limiting examples of phenol-containing compounds include carvatrol, eugenol, 4-hexyl resorcinol, bromchlorophene, thymol, and triclosan. Additional non-limiting examples of phenolic compounds include phenol and its homologs; mono- and polyalkyl, and aromatic halophenols; resorcinol and catechol, as well as their derivatives; and bisphenolic compounds. Examples of aromatic halophenols include ortho-chlorophenols, para-chlorophenols, para-bromophenols, and ortho-bromophenols. Additional non-limiting disclosure of individual phenol-containing compounds is found for example in U.S. Pat. No. 5,296,214, column 3, line 10, through column 4, line 32, the disclosure of which is hereby incorporated by reference.

Bisphenolic compounds include honokiol, magnolol, dehydrodieugenol, and their structural analogs. In one embodiment, the phenol-containing compound has a structure represented by formula (V): 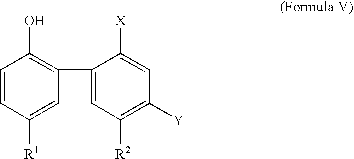
where one of X and Y is —OH and the other is —H, and R¹ and R² are independently C₁₋₈ alkyl or C₁₋₈ alkenyl. Examples include magnolol, honokiol, and their analogs. In another embodiment, the phenol-containing compound has a structure represented by formula (VI): 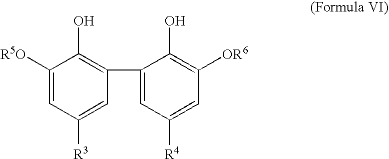
where R³ and R⁴ are independently C₁₋₈ alkyl or C₁₋₈ alkenyl, and R⁵ and R⁶ are independently C₁₋₄ alkyl, preferably methyl. Examples include dehydrodieugenol and structural analogs.

Compositions of the invention contain a phosphonate polymer, an amphoteric surfactant, and preferably a phenol-containing compound in amounts effective to inhibit the attachment of plaque bacteria to oral surfaces. In a convenient model, the action of inhibition of plaque formation on teeth is simulated by measuring the attachment of Actinomyces viscosus onto saliva-coated hydroxyl appetite beads according to standard methods.

In various embodiments, the oral composition is in a form suitable for application to an oral surface to prevent or inhibit bacterial film formation. Non-limiting examples of oral compositions include toothpastes or tooth gels, dentifrices, mouthwashes, mouthrinses, oral lozenges, chewing gums, edible strips, and the like. Depending on the physical form, the oral compositions contain conventional ingredients in addition to the phosphonate polymer, amphoteric surfactant, and preferably phenolic compound.

In addition to a biologically acceptable carrier, oral compositions of the invention preferably contain an effective amount of compound or compounds that inhibits the growth of oral bacteria. In some embodiments, such a compound is a phenol-containing compound as described above. Alternatively, the oral compositions contain antibacterial compounds other than the phenol-containing compound. In various embodiments, an antibacterial effective amount is from about 0.001% to about 10%, based on the total weight of the oral composition, for example from 0.01% to about 5% or about 0.1% to about 2%. The effective amount will vary depending on the form of the oral composition. For example, in tooth pastes, tooth gels, and tooth powders, an effective amount is usually at least about 0.01% and more preferably at least about 0.05%. In some preferred embodiments, an antibacterial compound is present in a tooth paste, gel, or powder at a level of 0.1% or more, to achieve a desired level of antibacterial activity. Normally, the antibacterial compound is formulated at 5% or less, preferably about 2% or less, and more preferably about 1% or less based on the total weight of the composition. Concentrations in the upper end of these limits can be used, but are sometimes less preferred for economic reasons. In various embodiments, optimum effectiveness is achieved at from about 0.1% to about 1%, especially from about 0.1% to about 0.5% or about 0.1% to about 0.3%, wherein all percentages are based on the total weight of the oral composition. Amounts used in tooth gels, tooth powders, gums, edible strips, and the like are comparable to those used in tooth pastes.

In mouth washes and rinses, an antibacterial effective amount is normally on the lower side of the above ranges. Typically, an antibacterial compound is used at a level of about 0.001% (or 10 ppm) up to about 1% or less, preferably about 0.5% or less or about 0.2% or less. Preferably it is about 0.01% (100 ppm) or greater. In various embodiments, oral compositions comprise from about 0.03 to about 0.12% by weight of an antibacterial compound.

In addition to the antibacterial compound, a number of active ingredients and functional materials are included in various compositions of the invention. Such materials include, without limitation, abrasives, humectants, surfactants, anticalculus agents, thickeners, viscosity modifiers, anticaries agents, flavorants, colorants, additional antibacterial agents, antioxidants, anti-inflammation components, and so on. They are added to the pastes, rinses, gums, lozenges, strips, and other forms of the oral compositions of the invention according to known methods.

In various embodiments, the oral composition preferably comprises a dentally acceptable abrasive material, which serves to either polish the tooth enamel or provide a whitening effect. Non-limiting examples include silica abrasives such as silica gels and precipitated silicas. Commercial embodiments include ZEODENT® 115, marketed by J. M. Huber and SYLODENT® XWA, SYLODENT® 783 or SYLODENT® 650 XWA of the Davison Chemical Division of W. R. Grace & Co. Other useful dentifrice abrasives include, without limitation, sodium metaphosphate, potassium metaphosphate, tricalcium phosphate, dihydrated dicalcium phosphate, aluminum silicate, calcined alumina, bentonite or other siliceous materials, or combinations thereof.

The composition of the invention may contain a humectant, useful for example to prevent hardening of a toothpaste upon exposure to air. Any orally acceptable humectant can be used, including without limitation polyhydric alcohols such as glycerin, sorbitol, xylitol and low molecular weight PEGs. Most humectants also function as sweeteners. One or more humectants are optionally present in a total amount of about 1% to about 70%, for example about 1% to about 50%, about 2% to about 25%, or about 5% to about 15% by weight of the composition.

The composition may contain surfactants other than the amphoteric surfactant that supplies a synergistic anti-plaque effect. Any orally acceptable surfactant, most of which are anionic, nonionic or amphoteric, can be used. Suitable anionic surfactants include without limitation water-soluble salts of C₈₋₂₀ alkyl sulfates, sulfonated monoglycerides of C₈₋₂₀ fatty acids, sarcosinates, taurates and the like. Illustrative examples of these and other classes include sodium lauryl sulfate, sodium coconut monoglyceride sulfonate, sodium lauryl sarcosinate, sodium lauryl isothionate, sodium laureth carboxylate and sodium dodecyl benzenesulfonate. Suitable nonionic surfactants include without limitation poloxamers, polyoxyethylene sorbitan esters, fatty alcohol ethoxylates, alkylphenol ethoxylates, tertiary amine oxides, tertiary phosphine oxides, and dialkyl sulfoxides. Suitable amphoteric surfactants include without limitation derivatives of C₈₋₂₀ aliphatic secondary and tertiary amines having an anionic group such as carboxylate, sulfate, sulfonate, phosphate or phosphonate. A suitable example is cocoamidopropyl betaine. One or more surfactants are optionally present in a total amount of about 0.01% to about 10%, for example about 0.05% to about 5% or about 0.1% to about 2% by weight of the composition.

The anti-calculus agent may be any know or to be developed in the art. One or more such agents can be present. Suitable anticalculus agents include without limitation phosphates and polyphosphates (for example pyrophosphates), polyaminopropanesulfonic acid (AMPS), zinc citrate trihydrate, polypeptides such as polyaspartic and polyglutamic acids, polyolefin sulfonates, polyolefin phosphates, diphosphonates such as azacycloalkane-2,2-diphosphonates (e.g., azacycloheptane-2,2-diphosphonic acid), N-methyl azacyclopentane-2,3-diphosphonic acid, ethane-1-hydroxy-1,1-diphosphonic acid (EHDP) and ethane-1-amino-1,1-diphosphonate, phosphonoalkane carboxylic acids and salts of any of these agents, for example their alkali metal and ammonium salts. Useful inorganic phosphate and polyphosphate salts illustratively include monobasic, dibasic and tribasic sodium phosphates, sodium tripolyphosphate (STPP), tetrapolyphosphate, mono-, di-, tri- and tetrasodium pyrophosphates, disodium dihydrogen pyrophosphate, sodium trimetaphosphate, sodium hexametaphosphate and the like, wherein sodium can optionally be replaced by potassium or ammonium. Other useful anticalculus agents include polycarboxylate polymers. These include polymers or copolymers of monomers that contain carboxylic acid groups, such as acrylic acid, methacrylic acid, and maleic acid or anhydride. Non-limiting examples include polyvinyl methyl ether/maleic anhydride (PVME/MA) copolymers, such as those available under the Gantrez™ brand from ISP, Wayne, N.J. Still other useful anticalculus agents include sequestering agents including hydroxycarboxylic acids such as citric, fumaric, malic, glutaric and oxalic acids and salts thereof, and aminopolycarboxylic acids such as ethylenediaminetetraacetic acid (EDTA). One or more anticalculus agents are optionally present in the composition in an anticalculus effective total amount, typically about 0.01% to about 50%, for example about 0.05% to about 25% or about 0.1% to about 15% by weight.

In another embodiment the composition comprises an orally acceptable source of fluoride ions. One or more such sources can be present. Suitable sources of fluoride ions include fluoride, monofluorophosphate and fluorosilicate salts, and amine fluorides, including olaflur (N′-octadecyltrimethylendiamine-N,N,N′-tris(2-ethanol)-dihydrofluoride). Any such salt that is orally acceptable can be used, including without limitation alkali metal (e.g., potassium, sodium), ammonium, stannous and indium salts and the like. Water-soluble fluoride-releasing salts are typically used. One or more fluoride-releasing salts are optionally present in an amount providing a total of about 100 to about 20,000 ppm, about 200 to about 5,000 ppm, or about 500 to about 2,500 ppm, fluoride ions. Where sodium fluoride is the sole fluoride-releasing salt present, illustratively an amount of about 0.01% to about 5%, about 0.05% to about 1% or about 0.1% to about 0.5%, sodium fluoride by weight can be present in the composition.

Other components include, without limitation, flavorants, colorants, and other active ingredients such as antioxidants and anti-inflammation agents. The components are formulated into oral compositions according to known procedures.

Tooth pastes and gels contain major amounts of humectants and usually an abrasive compound or compounds for teeth cleaning. They are formulated with various active ingredients, such as anticaries agents, anti-plaque compound, anti-inflammation agents, and the like, in addition to the antibacterial compound I.

Mouth rinses and mouth washes contain the active compound I in a liquid carrier such as water or water/ethanol. Generally, the compositions contain a major amount of solvent, up to 98 or 99% by weight. The active compound I is optionally formulated together with surfactants, colorants, flavorants, and other active ingredients.

In another embodiment, the invention provides for providing formation of plaque on an oral surface of an animal. The method comprises applying to the oral surface an oral composition that contains an amphoteric surfactant in combination with an ethylenic polymeric material comprising a plurality of phosphonate groups. The ethylenic polymeric material in various embodiments includes the phosphonate polymers described above. The composition preferably contains a betaine surfactant aand further contains a phenol-containing compound. In various embodiments, it is observed that the composition on application to an oral surface such as the teeth inhibits plaque formation on the surface to a higher extent than compositions lacking either the polymeric material or the amphoteric surfactants. The method can be carried out on humans or non-human subjects.

In another embodiment, the invention provides a method of improving or maintaining the systemic health of the subject. The method comprises applying to the oral cavity of the subject an oral composition containing an orally acceptable carrier, a betaine surfactant, a polymer comprising a plurality of phoshonate groups, and an antibacterial phenolic composition. Application of the composition to the oral cavity reduces the level of bacteria. In various embodiments, the polymer comprises any of the phosphonate polymers described above.

### EXAMPLES

### Example 1

### Mouthrinse Formulation

| Ingredient | Wt. % |
|---|---|
| Sorbitol | 10.0 |
| Tegobetaine (13% aq) | 1.42 |
| Glycerin | 10.0 |
| PVPA, Sodium salt | 3.61 |
| Ethanol (95% aq) | 10.0 |
| Propylene Glycol | 7.0 |
| Phenolic Flavor | 0.15 |
| Water to make (menthol) | 100.0 |

The above ingredients were combined into a mouthrinse that can be used twice daily.

### Example 2

### Toothpaste Formulation

| Ingredient | Wt. % |
|---|---|
| Glycerin | 25.0 |
| Carboxy methylcellulose | 1.3 |
| Saccharin | 0.3 |
| Sodium fluoride | 0.3 |
| Silica | 30.0 |
| Sodium laurylsulfate | 1.5 |
| Tegobetaine (30% aq) | 1.7 |
| Phenolic Flavor (menthol) | 1.0 |
| PVPA, sodium salt | 3.0 |
| Water to make | 100.0 |

The above ingredients were formulated into a toothpaste that can be used twice daily.

### Example 3

A mouthrinse formula of Example I is tested for percent plaque inhibition in a human clinical trial. It was compared to a similar formulation, but one which lacked betaine and the phenol-containing compound. For the mouthrinse of Example 1, 36% of plaque inhibition is observed. For the comparative mouthrinse, without the betaine and the phenol-containing compound, 21% inhibition of plaque is observed.

### Example 4

Various mouthrinse formulations are compared to the mouthrinse of Example 1. As in Example 6, the mouthrinse of Example 1 showed 36% inhibition of plaque.

A comparative example like Example 1 but containing 0.03 triclosan instead of the PVPA showed 26% reduction of plaque.

A mouthrinse as in Example 1 but lacking the PVPA and the phenolic flavor showed 19% inhibition of plaque.

A mouthrinse as in Example 1, but lacking the tegobetaine and phenolic flavor showed 21% inhibition of plaque.

A mouthrinse as in Example 1, but lacking the PVPA showed 13% inhibition of plaque.

We claim:

1. An oral composition comprising
an amphoteric surfactant; and
an homo- or copolymer comprising an ethylenic monomer that comprises a phosphonate group.

2. The composition according to claim 1, selected from the group consisting of a dentifrice, a mouth rinse, a lozenge, and a chewing gum.

3. The composition according to claim 1, wherein the amphoteric surfactant comprises a betaine surfactant.

4. The composition according to claim 1, wherein the polymer is a copolymer of vinyl phosphonic acid.

5. The composition according to claim 1, further comprising a phenol-containing compound.

6. An oral composition comprising
an amphoteric surfactant; and
a homo- or copolymer comprising a monomer represented by the structure of Formula (I): 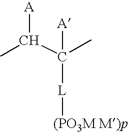
wherein
one of A and A′ is a hydrogen atom and the other is selected from a hydrogen atom or a group —(X)ₙRₘ; wherein n is i\selected from 0 or 1; provided that when n is 0, m is 1, and when n is 1, m is an integer of 1 to 3; X is selected from an oxygen atom, a sulfur atom, a nitrogen atom, a phosphorous atom, and silicon atom; R is independently a hyuderogen atom or an arganic radical;
L is selected from a bond and a linking group;
M and M′ are independently selected from a hydrogen atom, an alkali metal, and an ammonium ion, or together form an alkaline earth metal or other divalent element; and
p is 1 or 2, provided that when L is a bond, p is 1.

7. The composition of claim 6, wherein L represents a linking group selected from an alkylene radical or an alkyleneimino radical.

8. The composition according to claim 6, wherein the polymer has a number average molecular weight of at least about 1,000.

9. The composition according to claim 6, wherein the surfactant is a betaine surfactant.

10. The composition according to claim 6, wherein L is a bond.

11. The composition according to claim 6, wherein n is 0.

12. An oral composition comprising
a betaine surfactant;
a homo- or copolymer of vinyl phosphonic acid or a salt form of vinyl phosphonic acid.

13. The composition according to claim 12, wherein the homo- or copolymer comprises a polyvinyl phosphonic acid monomer.

14. The composition according to claim 12 further comprising a phenol-containing compound.

15. A method for inhibiting the adhesion of a bacterium on a dental surface comprising applying to the surface of the oral cavity an oral composition comprising an amphoteric surfactant and an homo- or copolymer comprising an ethylenic monomer that comprises a phosphonate group.

17. The method according to claim 16, wherein the ethylenic monomer that comprises a phosphonate group is present in the polymer in an amount of at least 50 mole %.

18. A method according to claim 16, wherein the wherein the ethylenic monomer that comprises a phosphonate group is present in the polymer in an amount of at least 90 mole %.

19. The method according to claim 17, wherein the amphoteric surfactant comprises a betaine surfactant.

20. The method according to claim 15, wherein the homo- or copolymer comprises a monomer represented by the structure of Formula (I): 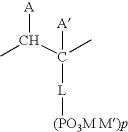
wherein
one of A and A′ is a hydrogen atom and the other is selected from a hydrogen atom or a group —(X)ₙRₘ; wherein n is i\selected from 0 or 1; provided that when n is 0, m is 1, and when n is 1, m is an integer of 1 to 3; X is selected from an oxygen atom, a sulfur atom, a nitrogen atom, a phosphorous atom, and silicon atom; R is independently a hydrogen atom or an organic radical;
L is selected from a bond and a linking group;
M and M′ are independently selected from a hydrogen atom, an alkali metal, and an ammonium ion, or together form an alkaline earth metal or other divalent element; and
p is 1 or 2, provided that when L is a bond, p is 1.

21. A method for promoting or improving the systemic heatlh of a human com[prisng applying to the surface or the oral cavity an oral composition comprising an amphoteric surfactant and an homo- or copolymer comprising an ethylenic monomer that comprises a phosphonate group.
